(19) United States
(12) Patent Application Publication
(10) Pub. No.: US 2007/0299450 A1
(43) Pub. Date: Dec. 27, 2007

(54) Pedicle Screw and Device for Injecting Bone Cement into Bone

(76) Inventors: Ji-Hoon Her, Kyunggi-do (KR); Sang-Hyoung Kim, Kyunggi-do (KR)

Correspondence Address: GREER, BURNS & CRAIN, 300 S WACKER DR, 25TH FLOOR, CHICAGO, IL, 60606, US

(21) Appl. No.: 11/794,443

(22) PCT Filed: Dec. 31, 2004
(86) PCT No.: PCT/KR04/03553

§ 371 Date: Aug. 10, 2007

(51) Int. Cl. A61B 17/58; A61B 17/56

(52) U.S. Cl. 606/73; 606/100; 606/99

(57) ABSTRACT

Disclosed is a pedicle screw used in spinal fusion surgery and a device for injecting bone cement into a spine having low bone mineral density by using the pedicle screw so as to enhance strength of the spine. The pedicle screw includes a screw rod fixedly inserted into a bone, a head section provided at an upper portion of the screw rod and formed at an inner portion thereof with a U-shaped recess and a screw part, and a coupling section coupled with the screw part of the head section. The coupling section is coupled with a rod support section including a bolt having a screw structure provided at an upper portion of the rod support section, a reverse U-shaped recess formed at a lower portion of the rod support section, and a pair of protrusions provided at lateral portions of the rod support section. The screw part of the head section is formed with a pair of guide slots. The screw rod is formed at an inner portion thereof with a hollow section, injection holes communicated with the hollow section are formed in the screw rod, and a feeding hole is formed in the U-shaped recess for feeding bone cement. The bone cement injection device has a cannula including an elongated body, a pedicle screw coupling member provided at one end of the elongated body so as to be fixed to the pedicle screw, and a handle provided at the other end of the elongated body and having an injector coupling member which is coupled to an inlet of an injector, and an impactor including an elongated cylindrical pressing rod inserted into the elongated body of the cannula and having a length longer than a length of the elongated body of the cannula, and a handle attached to one end of the elongated cylindrical pressing rod.

### TECHNICAL FIELD

The present invention relates to a pedicle screw used in spinal fusion surgery and a device for injecting bone cement into a spine having low bone mineral density by using the pedicle screw so as to enhance strength of the spine.

### BACKGROUND ART

In general, spinal cord injured patients who may hardly stand erect must undergo a surgical operation in order to implant an artificial aid into an injured spine for supporting the injured spine. Such an artificial aid for supporting the injured spine includes a pedicle screw installed at upper and lower portions of the injured spine for fixing the injured spine and a rod connected to the pedicle screw so as to support the injured spine.

FIG. 1 is a view illustrating a conventional pedicle screw implanted into a spine. As shown in FIG. 1, when a pedicle screw 2 and a rod 3 are implanted into the spine 1, a screw rod 4 of the pedicle screw 2 is inserted into the spine 1 in vertical to the spine 1 and the rod 3 is inserted into a head section 5 of the pedicle screw 2 in such a manner that the rod 3 can be rested in a U-shaped recess 5a of the head section 5 of the pedicle screw 3.

In this state, after aligning a fixing bolt 6 in line with an axis of a screw part 5b formed in the head section 5, a driver 7 is inserted into a groove 6a formed in an upper portion of the fixing bolt 6. Then, the driver 7 is rotated so as to screw-couple the fixing bolt 6 with the screw part 5b formed at an inner portion of the head section 5. Accordingly, the fixing bolt 6 presses the rod 3 accommodated in the head section 5 so that the rod 3 can securely support the spine 1.

However, the conventional pedicle screw 2 having the above structure presents a problem in that fixing force for the rod 3 is weak because the rod 3 is simply fixed by the fixing bolt 6. In order to solve the above problem, a ring is coupled with the rod 3 or a plate is inserted between the rod 3 and the fixing bolt 6 in order to enhance fixing force for the rod. However, such a structure is not adaptable for practical use because it is difficult to place the plate between the rod 3 and the fixing bolt 6 in a state that the rod 3 is accommodated in the head section 5 of the screw rod 4.

In order to implant the pedicle screw 2 into the spine, the spine must have sufficient strength. However, in cases of osteoporosis patients, an amount of calcium and collagen contained in bone may be insufficient, so a plurality of pores are formed in the bone and the bone becomes thin. For this reason, the bone may be easily broken even if relatively weak impact is applied to the bone. Thus, a pedicle screw 2 is implanted into the bone of osteoporosis patients. In this case, bone cement is injected into the bone of the osteoporosis patients so as to reinforce strength of the bone in such a manner that the bone can securely support the pedicle screw 2.

The bone cement is a material having a property substantially identical to that of the bone and is injected into the bone having a sparse internal structure so as to reinforce strength of the bone by filling up gaps formed in the bone. In general, a syringe or an injector is used for injecting the bone cement into the bone.

However, when the pedicle screw 2 is implanted into the spine 1 after injecting the bone cement into the spine 1, it is difficult to implant the pedicle screw 2 into the spine 1 due to increased strength of the spine 1. In addition, when the bone cement is injected into the spine 1 after the pedicle screw 2 has been implanted into the spine 1, it is difficult to inject the bone cement into the spine 1 around the pedicle screw 2.

### DISCLOSURE OF THE INVENTION

Therefore, the present invention has been made in view of the above-mentioned problems, and it is an object of the present invention to provide a pedicle screw which can be easily and stably implanted into bone at a time by means of a coupling section including a rod support section integrally formed with a fixing bolt capable of enhancing fixing force with respect to a rod, and which can be stably used for osteoporosis patients by injecting bone cement into bones of osteoporosis patients through a hollow section formed in a screw rod of the pedicle screw.

Another object of the present invention is to provide a device for easily injecting bone cement having relatively high density into bone around a pedicle screw implanted into the bone.

In order to accomplish the above objects, according to one aspect of the present invention, there is provided a pedicle screw comprising: a screw rod fixedly inserted into a bone; a head section provided at an upper portion of the screw rod and formed at an inner portion thereof with a U-shaped recess and a screw part; and a coupling section coupled with the screw part of the head section, wherein the coupling section is coupled with a rod support section including a bolt having a screw structure provided at an upper portion of the rod support section, a reverse U-shaped recess formed at a lower portion of the rod support section, and a pair of protrusions provided at lateral portions of the rod support section, and the screw part of the head section is formed with a pair of guide slots. The screw rod is formed at an inner portion thereof with a hollow section, injection holes communicated with the hollow section are formed in the screw rod, and a feeding hole is formed in the U-shaped recess for feeding bone cement.

In order to accomplish the above objects, according to another aspect of the present invention, there is provided a bone cement injection device comprising: a cannula including an elongated body, a pedicle screw coupling member provided at one end of the elongated body so as to be fixed to the pedicle screw, and a handle provided at the other end of the elongated body and having an injector coupling member which is coupled to an inlet of an injector; and an impactor including an elongated cylindrical pressing rod inserted into the elongated body of the cannula and having a length longer than a length of the elongated body of the cannula, and a handle attached to one end of the elongated cylindrical pressing rod.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a perspective view illustrating a conventional pedicle screw implanted into a spine;

FIG. 2 is an exploded perspective view illustrating a pedicle screw according to one embodiment of the present invention;

FIG. 3 is a sectional view taken along line “A-A′” shown in FIG. 2;

FIG. 4 is a perspective view of a cannula according to one embodiment of the present invention;

FIG. 5 is a perspective view of an impactor according to one embodiment of the present invention;

FIG. 6 is a sectional view of a bone cement injection device in which an impactor is inserted into a cannula according to one embodiment of the present invention;

FIG. 7 is a perspective view illustrating a cannula coupled with an injector for injecting bone cement into the cannula;

FIG. 8 is an operational view of a bone cement injection device according to one embodiment of the present invention; and

FIG. 9 is a view illustrating a modified embodiment of the present invention.

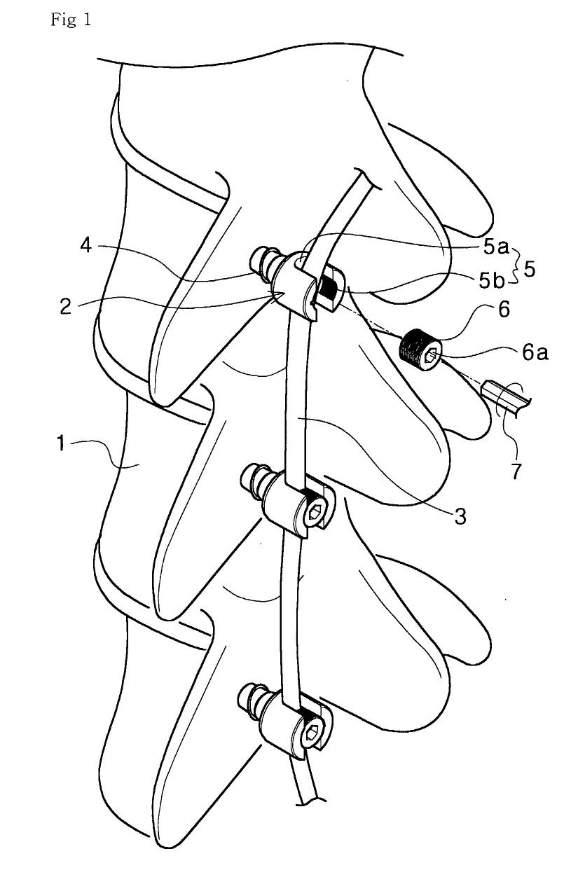

### BEST MODE FOR CARRYING OUT THE INVENTION

Reference will now be made in detail to the preferred embodiments of the present invention.

Hereinafter, a preferred embodiment of the present invention will be described with reference to accompanying drawings.

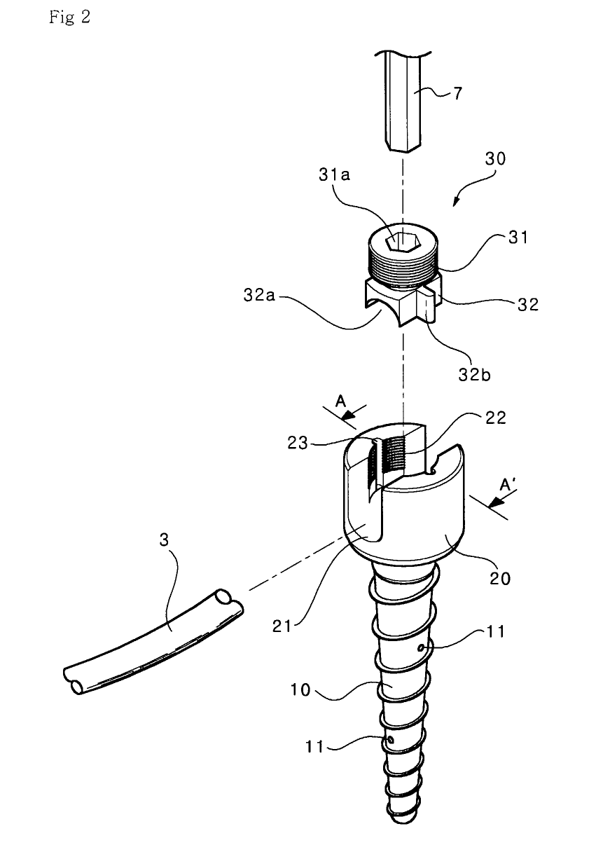

FIG. 2 is an exploded perspective view illustrating a pedicle screw according to one embodiment of the present invention.

As shown in FIG. 2, pedicle screw includes a screw rod 10, which is directly inserted into a spine. The screw rod 10 is formed at an outer surface thereof with a screw section. Injection holes 11 are formed in the screw rod 10 so as to inject bone cement into the spine.

The screw rod 10 is provided at an upper portion thereof with a head section 20, which is formed at an inner portion thereof with a U-shaped recess 21. When the pedicle screw is implanted into the spine, a rod 3 is rested in the U-shaped recess 21. The U-shaped recess 21 is formed with a feeding hole 12 for feeding bone cement. A screw part 22 is formed at an upper portion of the U-shaped recess 21 and a pair of guide slots 23 are formed in the screw part 22.

A coupling section 30 is coupled with the screw part 22. The coupling section 30 includes a bolt 31 having a screw-structure coupled with the screw part 22. The bolt 31 is formed at a center thereof with a hexagonal hole 31a, into which a driver 7 is inserted so as to rotate the bolt 21. In addition, a rod support section 32 is formed at a lower portion of the coupling section 30 in which a reverse U-shaped recess 32a is formed at a lower portion of the rod support section 32 and a pair of protrusions 32b are provided at lateral portions of the rod support section 32. The rod support section 32 is integrally formed with the bolt 31.

The rod 3 rested in the U-shaped recess 21 has a cylindrical shape. Since a lower-half part of the rod 3 is rested in the U-shaped recess 21 of the head section 20, a structure for supporting an upper-half part of the rod 3 is necessary in order to stably fix the rod 3. Thus, the reverse U-shaped recess 32a is formed at the lower portion of the rod support section 32 so as to prevent the rod 2 from slidably moving by securely making contact with the upper-half part of the rod 3.

When using the pedicle screw having the above structure, the screw rod 10 is implanted into the spine and the rod 3 is accommodated in the head section 20 formed at the upper portion of the screw rod 2 in such a manner that the rod 3 is securely rested in the U-shaped recess 21 of the head section 20. Then, in a state that the protrusions 32b of the rod support section 32 are inserted into the guide slots 23 of the head section 20, the driver 7 is inserted into the hexagonal hole 31a so as to rotate the coupling section 30. As the driver 7 inserted into the hexagonal hole 31a rotates, the bolt 31 formed at the upper portion of the coupling section 30 is screw-coupled with the screw part 22 formed at an inner portion of the head section, so that the reverse U-shaped recess 32a of the rod support section 32 is closely engaged with the upper-half part of the rod 3.

Therefore, according to the present invention having the rod support section 32 integrally formed with the bolt 31, it is not necessary to place the plate on the rod 3 and to fix the rod 3 by using the fixing bolt. In addition, since the upper-half part of the rod 3 is engaged with the reverse U-shaped recess 32 formed in the rod support section 32, the rod 3 can be securely fixed while being prevented from slidably moving.

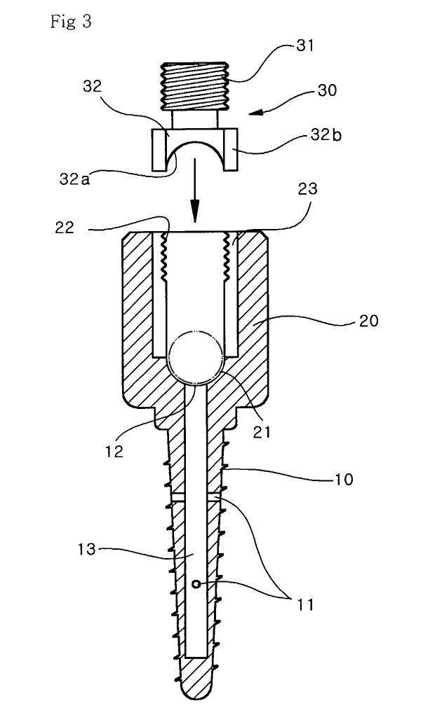

FIG. 3 is a sectional view taken along line “A-A′” shown in FIG. 2. As shown in FIG. 3, the screw bar 10 is formed at an inner portion thereof with a hollow section 13. In addition, the screw bar 10 is formed at an upper portion thereof with injection holes 11, which are formed in opposition to each other and communicated with each other through the hollow section 13 of the screw bar 10.

A circle shown in the U-shaped recess 21 of the head section 20 with a phantom line is a space in which the rod 3 is rested. The feeding hole 12 extends downward from the circle. That is, bone cement is injected into the feeding hole 12 by connecting a needle of the injector to the feeding hole 12 so that the bone cement fed into the hollow section 13 is injected into the bone through the injection holes 11 formed in the screw rod 10. After that the rod 3 is installed in the space.

When the bone cement has been injected into the bone through the above manner, the bone cement injected into the bone and the bone cement remaining the hollow section 13 may be solidified while being connected with each other. Thus, it is possible to more securely fix the spine as compared with a conventional method in which the bone cement is injected into the spine after forming a hole in the spine.

However, if the bone cement is injected into the pedicle screw according to the present invention by means of a general injection needle, since an inner diameter of the injection needle is small, the bone cement having high viscosity is rarely discharged through the injection needle. For this reason, when the bone cement is fed into the pedicle screw by using the injection needle, the bone cement having low viscosity must be used. The bone cement having low viscosity has superior fluidity, so it is possible to inject the bone cement into a required place through the injection needle. However, the bone cement may flow into other places from the required place. Since the bone cement may be injected into a predetermined portion of the bone in which pores are formed due to osteoporosis, if the bone cement flows through nerve holes of the spine, nerve palsy may occur. In addition, if the bone cement penetrates into a blood flow, the bone cement may flow into the lung or the heart, thereby causing embolism. To solve the above problem, the present invention suggests the bone cement injection device including a cannula and an impactor. Hereinafter, the bone cement injection device according to the present invention will be described in detail.

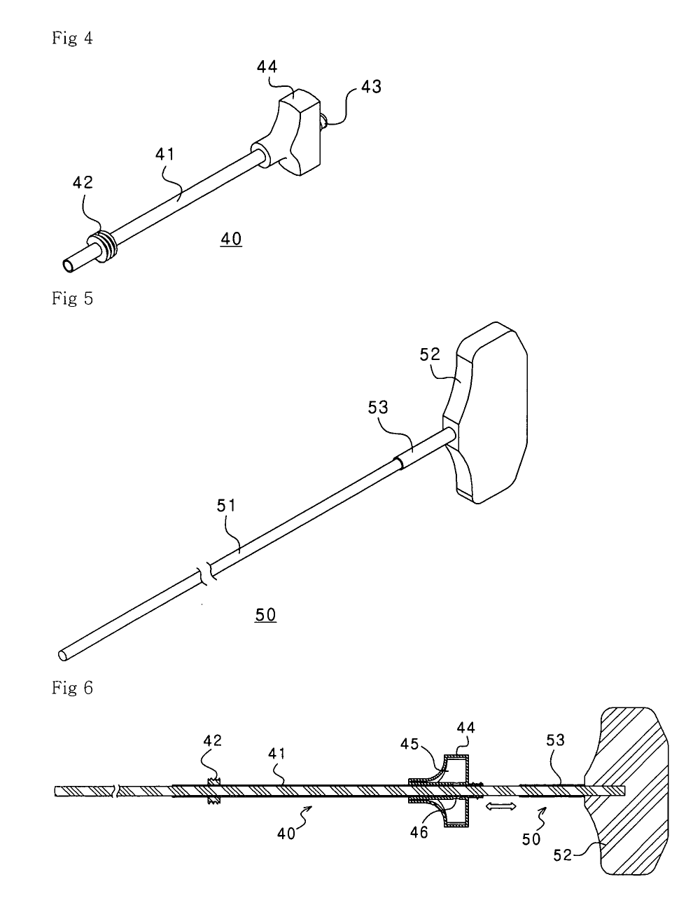

FIG. 4 is a perspective view of the cannula according to one embodiment of the present invention.

As shown in FIG. 4, the cannula 40 includes an elongated body 41, a pedicle screw coupling member 42 having a front end fixed to the pedicle screw, and a handle 44 provided with an injector coupling member 43 which is coupled to an inlet of the injector in order to fed the bone cement into the injector.

The elongated body 41 is made from a stainless material, which is harmless to humans. The pedicle screw coupling member 42 integrally formed with one end of the elongated body 41 includes a bolt having a size adapted to be coupled with a female screw formed in the pedicle screw. The front end of the pedicle screw coupling member 42 is slightly inserted into the head section 15 of the pedicle screw 20.

The handle 44 of the cannula 40 is provided at the other end of the elongated body 41 in opposition to the pedicle screw coupling member 42. The injector coupling member 43 is provided at a rear end of the handle 44 so as to couple the injector coupling member 43 with the injector when the bone cement is injected into the elongated body 41 of the cannula 40. According to the present invention, since the injector is formed at an outlet portion thereof with the female screw section coupled with the injector needle, the injector coupling member 43 includes a male screw section coupled with the female screw section of the injector.

In the meantime, the handle 44 and the injector coupling member 43 are formed at inner portions thereof with cylindrical holes aligned in line with a hollow section of the elongated body 41 of the cannula 40. In addition, as shown in FIG. 6, the handle 44 is formed at an inner portion thereof with a cavity 45 and a slot 46 communicated with the cylindrical hole of the elongated body 41 of the cannula 40. The slot 46 allows the bone cement to be collected in the cavity 45 without being leaked to an exterior if the bone cement backflows when the bone cement contained in the elongated body 41 of the cannula 40 is injected into the pedicle screw.

FIG. 5 is a perspective view of the impactor 50 according to one embodiment of the present invention.

The impactor 50 is used for injecting the bone cement contained in the elongated body 41 of the cannula 40 into the pedicle screw. The impactor 50 includes a pressing rod 51 inserted into the elongated body 41 of the cannula 40 in order to extrude the bone cement contained the elongated body 41 of the cannula 40 and a handle 52 for inserting the pressing rod 51 into the elongated body 41 of the cannula 40 by applying force to the pressing rod 51. Similarly to the elongated body 41 of the cannula 40, the pressing rod 51 is made from the stainless material, which is harmless to humans. An outer diameter of the pressing rod 51 is identical to or slightly smaller than an inner diameter 41 of the elongated body 41 of the cannula 40 in such a manner that the pressing rod 51 can smoothly move within the elongated body 41 of the cannula 40 while allowing air contained in the bone to be exhausted to the exterior when the bone cement of the elongated body 41 is extruded into the bone by means of the pressing rod 51. A length of the pressing rod 51 is slightly larger than the elongated body 41 of the cannula 40 so that both ends of the pressing rod 51 may extend out of the elongated body 41 of the cannula 40 when the pressing rod 51 is accommodated in the elongated body 41 of the cannula 40, thereby extruding the bone cement with sufficient pressing force.

In addition, a tube-type stopper 53 having a size larger than a thickness of a finger is attached to a rear end of the pressing rod 51. Accordingly, the finger of an operator cannot be caught between the handle 44 of the cannula 40 and the handle 52 of the impactor 50 or the handle 52 of the impactor 50 is prevented from making contact with the injector coupling member 43 of the cannula 40 by means of the stopper 53 when the pressing rod 51 is inserted into the elongated body 41 of the cannula 40 without injecting the bone cement into the elongated body 41 of the cannula 40.

FIG. 6 is a sectional view of the bone cement injection device in which the impactor is inserted into the cannula according to one embodiment of the present invention.

As shown in FIG. 6, the pressing rod 51 is accommodated in the elongated body 41 of the cannula 40. At this time, a fine gap is formed between an outer peripheral portion of the pressing rod 51 and an inner peripheral portion of the elongated body 41 of the cannula 40. Accordingly, most of the bone cement contained in the elongated body 41 of the cannula 40 is injected into the pedicle screw by means of the pressing rod 51, so that most of the bone cement is injected into the spine through the pedicle screw. At this time, even if some of the bone cement backflows from the spine due to an increase of pressure in the spine, the bone cement is collected in the cavity 45 through the slot 46 formed in the handle 44 of the cannula 40 without being leaked out of the injector coupling member 43 provided at the rear portion of the cannula 40.

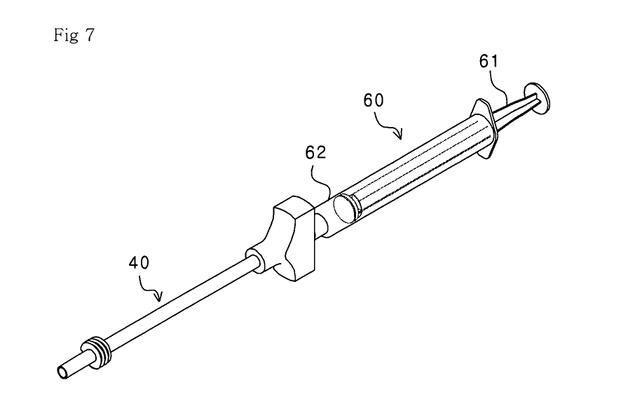

FIG. 7 is a perspective view illustrating the cannula coupled with the injector for injecting the bone cement into the cannula.

In order to inject the bone cement into the spine through the pedicle screw according to the present invention, the injector 60 sucks the bone cement and injects the bone cement into the cannula 40. To this end, an outlet of the injector 60 filled up with the bone cement is coupled to the injector coupling member 43 provided at the rear portion of the handle 44 of the cannula 40. In this state, a plunger 61 is pressed so as to inject the bone cement contained in a barrel 62 of the injector 60 into the cannula 40.

At this time, since the bone cement injected into the cannula 40 has high viscosity, the bone cement contained in the cannula 40 does not flow to the exterior even if the cannula 40 is horizontally aligned after decoupling the injector 60 from the cannula 40.

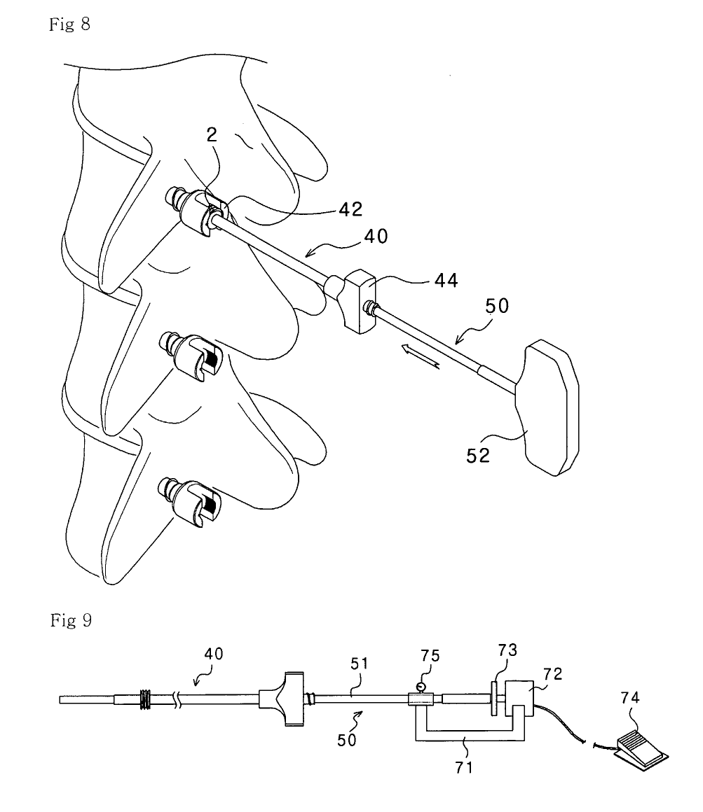

FIG. 8 is an operational view of the bone cement injection device according to one embodiment of the present invention.

As is described with reference to FIG. 7, after injecting the bone cement into the cannula 40 by using the injector 60, the cannula 40 is coupled with the pedicle screw 2 fixedly implanted into the spine. At this time, a front end of the cannula 40 is inserted into an opening of the pedicle screw and the pedicle screw coupling member 42 is coupled with the U-shaped recess and the screw part of the pedicle screw 2.

After that, the pressing rod 51 of the impactor 50 is inserted into the elongated body 41 of the cannula 40. In this state, external force is applied to the handle 52 of the impactor 50 in a longitudinal direction of the impactor 50, so that the bone cement contained in the elongated body 41 of the cannula 40 is injected into the spine.

In addition, the present invention can be variously modified. For instance, as shown in FIG. 9, the pressing rod 51 is fixed by means of a fixing unit 71 having a “ [symbol] ”-shape. In this state, a pressing unit 73 driven by a power source 72 presses the end of the pressing rod 51 so that the pressing rod 51 moves into the cannula 40, thereby automatically injecting the bone cement into the spine. Herein, the power source 72 is operated when the operator steps on a pedal 74. At this time, the operator may operate the power source 72 while checking a pressure gauge 75 in order to check whether a predetermined amount of the bone cement (about 0.1 cc whenever the operator steps on the pedal 74) is injected into the spine.

### INDUSTRIAL APPLICABILITY

As can be seen from the foregoing, according to the present invention, the pedicle screw can be easily and stably implanted into the bone at a time by means of the coupling section including the rod support section integrally formed with the fixing bolt. In addition, the reverse U-shaped recess is formed at the lower portion of the rod support section in such a manner that the reverse-U-shaped recess makes contact with the upper-half part of the rod, so the rod is prevented from slidably moving on the screw rod. Since the bone cement is fed into the cavity formed in the screw rod, the present invention is applicable for patients having weak bonds due to various diseases, such as osteoporosis patients.

In addition, according to the bone cement injection device of the present invention, the bone cement is injected into the spine through the pedicle screw implanted into the spine, so strength of the spine around the pedicle screw can be effectively reinforced. Furthermore, sufficient pressing force is applied to the bone cement contained in the cannula by means of the impactor, so the present invention can employ the bone cement having high viscosity. Even if the bone cement backflows from the spine while the bone cement is being injected into the spine, the back-flowed bone cement is collected in the cavity of the handle attached to the cannula without being leaked to the exterior, thereby preventing side effect caused by the bone cement being leaked.

While this invention has been described in connection with what is presently considered to be the most practical and preferred embodiment, it is to be understood that the invention is not limited to the disclosed embodiment and the drawings, but, on the contrary, it is intended to cover various modifications and variations within the spirit and scope of the appended claims.

1. A pedicle screw comprising:
a screw rod fixedly inserted into a bone;
a head section provided at an upper portion of the screw rod and formed at an inner portion thereof with a U-shaped recess and a screw part; and
a coupling section coupled with the screw part of the head section, wherein the screw rod is formed at an inner portion thereof with a hollow section, injection holes communicated with the hollow section are formed in the screw rod, and a feeding hole for feeding bone cement is formed in the U-shaped recess.

2. A pedicle screw comprising:
a screw rod fixedly inserted into a bone;
a head section provided at an upper portion of the screw rod and formed at an inner portion thereof with a U-shaped recess and a screw part; and
a coupling section coupled with the screw part of the head section, wherein the coupling section is coupled with a rod support section including a bolt having a screw structure provided at an upper portion of the rod support section, a reverse U-shaped recess formed at a lower portion of the rod support section, and a pair of protrusions provided at lateral portions of the rod support section, and the screw part of the head section is formed with a pair of guide slots.

3. The pedicle screw as claimed in claim 2, wherein the screw rod is formed at an inner portion thereof with a hollow section, injection holes communicated with the hollow section are formed in the screw rod, and a feeding hole is formed in the U-shaped recess for feeding bone cement.

4. The pedicle screw as claimed in claim 3, wherein the injection holes are formed at upper and lower portions of the screw rods in opposition to each other while communicating with the hollow section.

5. A bone cement injection device comprising:
a cannula including an elongated body, a pedicle screw coupling member provided at one end of the elongated body so as to be fixed to the pedicle screw, and a handle provided at the other end of the elongated body and having an injector coupling member which is coupled to an inlet of an injector; and
an impactor including an elongated cylindrical pressing rod inserted into the elongated body of the cannula and having a length longer than a length of the elongated body of the cannula, and a handle attached to one end of the elongated cylindrical pressing rod.

6. The bone cement injection device as claimed in claim 5, wherein the handle of the cannula is formed at an inner portion thereof with a cavity communicated with the elongated body of the cannula.

7. The bone cement injection device as claimed in claim 5, wherein the elongated cylindrical pressing rod is provided with a stopper in adjacent to the handle of the impactor so as to limit a movement of the elongated cylindrical pressing rod inserted into the elongated body of the cannula.

8. The bone cement injection device as claimed in claim 5, wherein the elongated body of the cannula and the elongated cylindrical pressing rod of the impactor are made from stainless materials, which are harmless to humans.
